(19) United States
(12) Patent Application Publication
(10) Pub. No.: US 2008/0008674 A1
(43) Pub. Date: Jan. 10, 2008

(54) Use of C-glycoside derivative for improving the skin's barrier function

(75) Inventors: Veronique Burnier, Draveil (FR); Isabelle Besne, Puteaux (FR)

Correspondence Address: OLIFF & BERRIDGE, PLC, P.O. BOX 320850, ALEXANDRIA, VA, 22320-4850, US

(73) Assignee: L'OREAL, PARIS (FR)

(21) Appl. No.: 11/822,114

(22) Filed: Jul. 2, 2007

Related U.S. Application Data
(60) Provisional application No. 60/836,659, filed on Aug. 10, 2006.

(30) Foreign Application Priority Data
Jul. 3, 2006 (FR) 06 06025

(51) Int. Cl. A61K 8/73

(52) U.S. Cl. 424/70.13

(57) ABSTRACT

The present invention relates to a cosmetic method for improving and/or reinforcing the skin's barrier function comprising at least a step of applying to the skin a composition comprising at least one C-glycoside derivative.

This non provisional application claims the benefit of French Application No. 06 06025 filed on Jul. 3, 2006 and U.S. Provisional Application No. 60/836,659 filed on Aug. 10, 2006.

The present invention relates to a cosmetic method for improving and/or reinforcing the skin's barrier function comprising at least a step of applying to the skin a composition comprising at least one C-glycoside derivative. The invention also relates to a cosmetic skin treatment process for improving the barrier function, comprising at least one step that consists in applying to the skin at least one composition comprising at least one C-glycoside derivative.

Human skin consists of two compartments, namely a deep compartment, the dermis, and a surface compartment, the epidermis.

It constitutes a barrier against external attack, especially chemical, mechanical or infectious attack, and, in this respect, a certain number of defence reactions against environmental factors (climate, ultraviolet rays, tobacco, etc.) and/or xenobiotics, for instance microorganisms, take place therein. This property, known as the barrier function, is mainly performed by the uppermost layer of the epidermis, i.e. the horny layer, known as the stratum corneum.

The cells constituting the epidermis (mainly the keratinocytes, but also the melanocytes and the Langerhans cells) are delimited by an intercellular lipid structure. Each of these cell types contributes by virtue of its intrinsic functions towards the essential role played in the body by the skin. In particular, keratinocytes undergo a continuous and directed maturation process, which, from the keratinocytes present in the basal layer of the epidermis, results in the formation of comeocytes, which are totally keratinized dead cells consisting of keratinocytes at the final stage of their differentiation.

In the course of differentiation, the phospholipids, whose role consists in producing the fluid structure of the cell membranes of the live layers of the epidermis, are gradually replaced by a mixture predominantly composed of fatty acids, cholesterol and sphingolipids (ceramides). These lipids, which are organized in specific lamellar liquid-crystal phases, form the intracellular cement of the stratum corneum and are essential for the water exchanges and the barrier function of the epidermis. Thus, the lamellar structure of the lipids in the lipid domain of the epidermis and the corneocytes participate in the epidermal barrier function.

It is quite obvious that the quality of the barrier function and of the mucous membranes is dependent on complex endogenous biological mechanisms involving numerous growth factors, adhesion molecules, hormones and lipid metabolism enzymes.

Thus, impairment of the skin barrier may take place in the presence of external attacking factors such as irritants (detergents, acids, bases, oxidizing agents, reducing agents, concentrated solvents, and toxic gases or fumes), mechanical stresses (friction, impacts, abrasion, tearing of the surface, projection of dust or particles, shaving or hair removal), thermal or climatic imbalances (cold, dryness, radiations), xenobiotics (undesirable microorganisms, allergens) or internal attacking factors such as psychological stress.

This impairment in the skin barrier may especially be reflected by cutaneous discomfort, sensory phenomena and especially unpleasant phenomena. The sensory nerve endings of the surface of the skin or the scalp, especially the C fibres of neurons, the chemical, heat or mechanical receptors or sensors or the pressure receptors are, in effect, partially “denuded”. The person may then experience a sensation of cutaneous discomfort that may be manifested especially by stinging, tautness, sensations of heating, or itching.

These sensations of cutaneous discomfort are more common in the most exposed areas of the body, namely the hands, the feet, the face and the scalp.

They may especially occur on areas subjected to certain daily or frequently repeated hygiene actions such as shaving, hair removal, cleansing with personal hygiene products or household products, the application of adhesives (dressings, patches, or the attachment of prostheses) or in the case of sports-related or occupational actions or actions simply associated with the lifestyle and with the use of clothing, tools or equipment that give rise to localized friction. They may also be amplified by psychological stress.

These sensations of cutaneous discomfort affect all people, and in particular:
people with “fragile” or “delicate” and vulnerable skin that rapidly becomes imbalanced during large changes in temperature or relative humidity (for example in the case of baby skin);
people with “depleted” skin, especially including
   people whose protective hydrolipid film composed of sweat, sebum and natural moisturizing factors becomes rarefied, as is the case for people over 60 years old and especially in the case of the very elderly (at least 75 years old);
   people for whom the composition of the hydrolipid film is modified, as is the case for diabetics or people undergoing dialysis treatment, or people suffering from certain diseases;
people with a lowered reactivity threshold due to neurogenic hyperactivity; these skin types will thus show these sensations and these clinical signs much more quickly and more frequently than other skin types: these are people with sensitive skin.

Mention may also be made of people with “aggravated” skin, for example for shaved skin.

It is moreover known that atopic skin types may be associated with a reduction in the synthesis of ceramides 1 and/or 3, as is especially reported in the following documents:
Imokawa et al., in J. Invest. Dermatol., 96 (4): 523-6, 1991, who describe that ceramides are involved in the barrier function and that their synthesis, in particular that of ceramide 1, is reduced in the case of atopic and xerotic skin types;
Di Nardo et al., in Acta Derm. Venereol., 78 (1): 27-30 (1998), who describe atopic dermatitis as being an easily irritable and dry skin, in which the barrier function is impaired.

It is thus sought to improve and/or reinforce the skin's barrier function in order:
to prevent and/or reduce the sensations of cutaneous discomfort, stinging, tautness, sensations of heating and itching, in particular in the case of people with fragile or delicate skin (for example babies); or people with depleted skin (such as people at least 60 years old and in particular people at least 75 years old), or people for whom the composition of the hydrolipid film is modified, as is the case for diabetics or people undergoing dialysis treatment, or people suffering from certain diseases, or people with sensitive skin;
and/or to improve the skin's barrier function for atopic skin types and/or to prolong the remission phases between acute crises of complaints of this type.

In this context, the inventors have discovered that certain C-glycoside derivatives are capable of improving and/or reinforcing the skin's barrier function.

Sugars and sugar derivatives are products that are already exploited for various purposes for the formulation of cosmetic compositions intended either for skincare or for caring for and/or washing keratin fibres.

Thus, in document U.S. Pat. No. 6,495,147, D-xylose and derivatives thereof are proposed for the purpose of preparing cosmetic or pharmaceutical products for improving the functionality of epidermal cells.

Among the sugars that may be used in the field, C-glycoside derivatives prove to be most particularly advantageous. Certain C-glycoside derivatives have especially demonstrated advantageous biological properties, in particular for combating epidermal ageing and/or skin dryness. Such compounds are especially described in document U.S. Pat. No. 7,049,300.

These compounds act by stimulating the synthesis of glycosaminoglycans containing a D-glucosamine and/or N-acetyl-D-glucosamine residue and are represented by the formula: 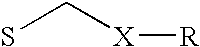
in which S represents a monosaccharide or a polysaccharide, R represents various linear or cyclic radicals and the group X may represent a group chosen from: —CO—, —CH(NR₁R₂)—, —CHR′, —C(═CHR₃)— with R₁, R₂, R′ and R₃ possibly representing various radicals, including the hydroxyl radical for R₁, R₂ and R₃.

However, these derivatives have hitherto never been used for the purposes of improving and/or reinforcing the skin's barrier function.

By means of this improvement and/or reinforcement of the skin's barrier function via the topical application of a composition according to the invention, all skin types, and in particular fragile or depleted skin (for example baby skin, the skin of people at least 60 years old and preferably at least 75 years old, the skin of diabetics or the skin of people undergoing dialysis treatment) and sensitive skin, are better protected against chemical, mechanical or infectious external attack.

The inventors have in particular demonstrated on a model of reconstructed skin an improvement in the barrier function of the skin by reducing the passage of a radiolabelled caffeine after treating the said skin with the said C-glycoside compounds.

According to a first of its aspects, the invention thus relates to a cosmetic method for improving the skin's barrier function using a C-glycoside derivative.

According to another of its aspects, the invention also relates to a cosmetic method for improving and/or reinforcing the protection of the skin against external attack comprising at least a step of applying to the skin a composition comprising at least one C-glycoside derivative.

In particular, the said C-glycoside derivative and/or the said composition may be intended for preventing and/or reducing the cutaneous discomfort of a skin type, in particular induced by exogenous stress of chemical, environmental or mechanical origin and/or endogenous stress, in particular of a fragile or depleted skin and/or of a sensitive skin, as defined above.

Cutaneous discomfort may especially be characterized by tautness, stinging, sensations of heating and/or itching.

According to another embodiment, the said C-glycoside derivative and/or the said composition may be intended for improving and/or reinforcing the barrier function of a skin type chosen from fragile skin, depleted skin, aggravated skin and/or sensitive skin.

In the context of the invention, the C-glycoside derivatives may be used for application to healthy skin, that is subjected or that may be subjected to the influence of agents such as climatic factors and as a result be liable to exhibit cutaneous discomfort. In other particular cases, the C-glycoside derivatives of the invention may be applied to the skin when it shows clinical signs of deficiency of the barrier function, for example atopic skin types.

Thus, one subject of the invention is the use of at least one C-glycoside derivative for the preparation of a composition, especially a dermatological composition, for improving and/or reinforcing the skin's barrier function.

Another aspect of the present invention is thus a dermatological method for improving and/or reinforcing the barrier function of damaged skin, in particular of atopic skin, comprising at least a step of applying to the skin a composition comprising at least one C-glycoside derivative.

The composition according to the invention may especially be intended for prolonging the remission phases between acute crises of dermatological complaints, for example of atopic type.

C-Glycoside Derivatives

A C-glycoside derivative that is suitable for use in the invention may be a compound of general formula (I) below: 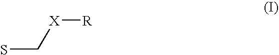
in which:

R represents:
a saturated C₁-C₂₀ and in particular C₁-C₁₀ or unsaturated C₂-C₂₀ and in particular C₂-C₁₀ linear alkyl radical, or a saturated or unsaturated, branched or cyclic C₃-C₂₀ and in particular C₃-C₁₀ alkyl radical;
a saturated C₁-C₂₀ and in particular C₁-C₁₀ or unsaturated C₂-C₂₀ and in particular C₂-C₁₀, or saturated or unsaturated, branched or cyclic C₃-C₂₀ and in particular C₃-C₁₀ linear hydrofluoroalkyl or perfluoroalkyl radical;
   the hydrocarbon-based chain constituting the said radicals possibly being, where appropriate, interrupted with 1, 2, 3 or more heteroatoms chosen from:
an oxygen,
a sulfur,
a nitrogen, and
a silicon,
   and possibly being optionally substituted with at least one radical chosen from:
—OR₄,
—SR₄,
—NR₄R₅,
—COOR₄,
—CONHR₄,
—CN,
a halogen atom,
a C₁-C₆ hydrofluoroalkyl or perfluoroalkyl radical, and/or
a C₃-C₈ cycloalkyl radical,
   with R₄ and R₅ possibly representing, independently of each other, a hydrogen atom or a saturated C₁-C₃₀ and in particular C₁-C₁₂ or unsaturated C₂-C₃₀ and in particular C₂-C₁₂, or a saturated or unsaturated, branched or cyclic C₃-C₃₀ and in particular C₃-C₁₂ linear alkyl, perfluoroalkyl or hydrofluoroalkyl radical; or a C₆-C₁₀ aryl radical,

X represents a radical chosen from the groups: 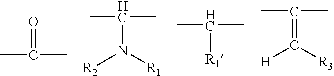
with R₁, R₂ and R₃ representing, independently of each other, a hydrogen atom or a radical R, with R as defined above, and R′₁ represents a hydrogen atom, an —OH group or a radical R as defined above, R₁ possibly also denoting a C₆-C₁₀ aryl radical;

S represents a monosaccharide or a polysaccharide comprising up to 20 sugar units and in particular up to 6 sugar units, in pyranose and/or furanose form and of L and/or D series, the said mono- or polysaccharide possibly being substituted with a mandatorily free hydroxyl group, and optionally one or more optionally protected amine function(s), and

the bond S—CH₂—X represents a bond of C-anomeric nature, which may be α or β, and also the cosmetically acceptable salts thereof, the solvates thereof such as hydrates, and the isomers thereof.

In the context of the present invention, the term “halogen” means chlorine, fluorine, bromine or iodine.

The term “aryl” denotes an aromatic ring such as phenyl, optionally substituted with one or more C₁-C₄ alkyl radicals.

The term “C₃-C₈ cycloalkyl” denotes an aliphatic ring containing from 3 to 8 carbon atoms, for example including cyclopropyl, cyclopentyl and cyclohexyl.

Among the alkyl groups that are suitable for use in the invention, mention may be made especially of methyl, ethyl, isopropyl, n-propyl, n-butyl, t-butyl, isobutyl, sec-butyl, pentyl, n-hexyl, cyclopropyl, cyclopentyl, cyclohexyl and allyl groups.

According to one embodiment of the invention, it is possible to use a C-glycoside derivative corresponding to formula (I) for which S may represent a monosaccharide or a polysaccharide containing up to 6 sugar units, in pyranose and/or furanose form and of L and/or D series, the said monosaccharide or polysaccharide containing at least one hydroxyl function that is mandatorily free and/or optionally one or more amine functions that are mandatorily protected, X and R otherwise retaining all the definitions given above.

Advantageously, a monosaccharide of the invention may be chosen from D-glucose, D-galactose, D-mannose, D-xylose, D-lyxose, L-fucose, L-arabinose, L-rhamnose, D-glucuronic acid, D-galacturonic acid, D-iduronic acid, N-acetyl-D-glucosamine and N-acetyl-D-galactosamine, and advantageously denotes D-glucose, D-xylose, N-acetyl-D-glucosamine or L-fucose, and in particular D-xylose.

More particularly, a polysaccharide of the invention containing up to 6 sugar units may be chosen from D-maltose, D-lactose, D-cellobiose, D-maltotriose, a disaccharide combining a uronic acid chosen from D-iduronic acid and D-glucuronic acid with a hexosamine chosen from D-galactosamine, D-glucosamine, N-acetyl-D-galactosamine and N-acetyl-D-glucosamine, an oligosaccharide containing at least one xylose advantageously chosen from xylobiose, methyl-β-xylobioside, xylotriose, xylotetraose, xylopentaose and xylohexaose and especially xylobiose, which is composed of two xylose molecules linked via a 1-4 bond.

More particularly, S may represent a monosaccharide chosen from D-glucose, D-xylose, L-fucose, D-galactose and D-maltose, especially D-xylose.

According to another embodiment of the invention, it is possible to use C-glycoside derivatives corresponding to formula (I) for which X represents a group chosen from —CO—, —CH(OH)—, —CH(NR₁R₂)— and —CH(R)—, in particular —CO—, —CH(OH)—, —CH(NH₂)—, —CH(NHCH₂CH₂CH₂OH)—, —CH(NHPh)— and —CH(CH₃)—, and more particularly a —CO—, —CH(OH)— or —CH(NH₂)— group, and preferentially a —CH(OH)— group, S and R otherwise conserving all of the definitions given above.

According to another embodiment of the invention, it is possible to use a C-glycoside derivative corresponding to formula (I) for which R represents a saturated C₁-C₂₀ and in particular C₁-C₁₀ or unsaturated C₂-C₂₀ and in particular C₂-C₁₀ linear alkyl radical, or a saturated or unsaturated, branched or cyclic C₃-C₂₀ and in particular C₃-C₁₀ alkyl radical; and optionally substituted as described above, S and X otherwise conserving all the definitions given above. Preferably, R denotes a linear C₁-C₄ and especially C₁-C₃ radical, optionally substituted with —OH, —COOH or —COOR″₂, R″₂ being a saturated C₁-C₄ alkyl radical, especially ethyl.

Preferentially, R denotes an unsubstituted linear C₁-C₄ and especially C₁-C₂ alkyl radical, in particular ethyl.

Among the C-glycoside derivatives of formula (I) that are preferably used are those for which:
R represents a saturated C₁-C₂₀ and in particular C₁-C₁₀ or unsaturated C₂-C₂₀ and in particular C₂-C₁₀ linear alkyl radical, or a saturated or unsaturated, branched or cyclic C₃-C₂₀ and in particular C₃-C₁₀ alkyl radical, optionally substituted as described above;
S represents a monosaccharide as described above;
X represents —CO—, —CH(OH)—, —CH(NR₁R₂)— or —CH(R)—, as defined above.

Preferably, a C-glycoside derivative of formula (I) is used, for which:
R denotes a linear C₁-C₄ and especially C₁-C₃ radical, optionally substituted with —OH, —COOH or —COOR″₂, R″₂ being a saturated C₁-C₄ alkyl radical, especially ethyl;
S represents a monosaccharide as described above;
X represents a group chosen from —CO—, —CH(OH)—, —CH(NH₂)—, —CH(NHCH₂CH₂CH₂OH)—, —CH(NHPh)- and —CH(CH₃)—, and more particularly a —CO—, —CH(OH)— or —CH(NH₂)— group, and preferentially a —CH(OH)— group.

Preferentially, a C-glycoside derivative of formula (I) is used, for which:
R denotes an unsubstituted linear C₁-C₄ and especially C₁-C₂ alkyl radical, in particular ethyl;
S represents a monosaccharide as described above; especially D-glucose, D-xylose, N-acetyl-D-glucosamine or L-fucose, in particular D-xylose;
X represents a group chosen from —CO—, —CH(OH)— and —CH(NH₂)— and preferentially a CH(OH)— group.

The salts that are acceptable for the non-therapeutic use of the compounds described in the present invention comprise conventional non-toxic salts of the said compounds such as those formed from organic or inorganic acids. Examples that may be mentioned include the salts of mineral acids, such as sulfuric acid, hydrochloric acid, hydrobromic acid, hydriodic acid, phosphoric acid or boric acid. Mention may also be made of the salts of organic acids, which may comprise one or more carboxylic, sulfonic or phosphonic groups. They may be linear, branched or cyclic aliphatic acids or alternatively aromatic acids. These acids may also comprise one or more heteroatoms chosen from O and N, for example in the form of hydroxyl groups. Mention may be made especially of propionic acid, acetic acid, terephthalic acid, citric acid and tartaric acid.

When the compound of formula (I) comprises an acid group, neutralization of the acid group(s) may be performed with a mineral base, such as LiOH, NaOH, KOH, Ca(OH)₂, NH₄OH, Mg(OH)₂ or Zn(OH)₂; or with an organic base such as a primary, secondary or tertiary alkylamine, for example triethylamine or butylamine. This primary, secondary or tertiary alkylamine may comprise one or more nitrogen and/or oxygen atoms and may thus comprise, for example, one or more alcohol functions; mention may be made especially of amino-2-methyl-2-propanol, triethanolamine, dimethylamino-2-propanol or 2-amino-2-(hydroxymethyl)-1,3-propanediol. Mention may also be made of lysine or 3-(dimethylamino)propylamine.

The solvates that are acceptable for the compounds described in the present invention comprise conventional solvates such as those formed during the final step of preparation of the said compounds due to the presence of solvents. Examples that may be mentioned include the solvates due to the presence of water or of linear or branched alcohols, for instance ethanol or isopropanol.

Among the C-glycoside derivatives of formula (I) used according to the invention, the ones that are most particularly considered are:
1. C-β-D-xylopyranoside-n-propan-2-one;
2. C-α-D-xylopyranoside-n-propan-2-one;
3. 1-[2-(3-hydroxypropylamino)propyl]-C-β-D-xylopyranose;
4. 1-[2-(3-hydroxypropylamino)propyl]-C-α-D-xylopyranose;
5. C-β-D-xylopyranoside-2-hydroproxyopane;
6. C-α-D-xylopyranoside-2-hydroxypropane;
7. C-β-D-xylopyranoside-2-aminopropane;
8. C-α-D-xylopyranoside-2-aminopropane;
9. C-β-D-xylopyranoside-2-phenylaminopropane;
10. C-α-D-xylopyranoside-2-phenylaminopropane;
11. ethyl 3-methyl-4-(C-β-D-xylopyranoside)butyrate;
12. ethyl 3-methyl-4-(C-α-D-xylopyranoside)butyrate;
13. 6-(C-β-D-xylopyranoside)-5-ketohexanoic acid;
14. 6-(C-α-D-xylopyranoside)-5-ketohexanoic acid;
15. 6-(C-β-D-xylopyranoside)-5-hydroxyhexanoic acid;
16. 6-(C-α-D-xylopyranoside)-5-hydroxyhexanoic acid;
17. 6-(C-β-D-xylopyranoside)-5-aminohexanoic acid;
18. 6-(C-α-D-xylopyranoside)-5-aminohexanoic acid;
19. 6-(C-β-D-xylopyranoside)-5-phenylaminohexanoic acid;
20. 6-(C-α-D-xylopyranoside)-5-phenylaminohexanoic acid;
21. 1-(C-β-D-xylopyranoside)hexane-2,6-diol;
22. 1-(C-α-D-xylopyranoside)hexane-2,6-diol;
23. 5-(C-β-D-xylopyranoside)-4-ketopentanoic acid;
24. 5-(C-α-D-xylopyranoside)-4-ketopentanoic acid;
25. 5-(C-β-D-xylopyranoside)-4-hydroxypentanoic acid;
26. 5-(C-β-D-xylopyranoside)-4-hydroxypentanoic acid;
27. 5-(C-β-D-xylopyranoside)-4-aminopentanoic acid;
28. 5-(C-α-D-xylopyranoside)-4-aminopentanoic acid;
29. 5-(C-β-D-xylopyranoside)-4-phenylaminopentanoic acid;
30. 5-(C-α-D-xylopyranoside)-4-phenylaminopentanoic acid;
31. 1-(C-β-D-xylopyranoside)pentane-2,5-diol;
32. 1-(C-α-D-xylopyranoside)pentane-2,5-diol;
33. 1-(C-β-D-fucopyranoside)propan-2-one;
34. 1-(C-α-D-fucopyranoside)propan-2-one;
35. 1-(C-β-L-fucopyranoside)propan-2-one;
36. 1-(C-α-L-fucopyranoside)propan-2-one;
37. 1-(C-β-D-fucopyranoside)-2-hydroxypropane;
38. 1-(C-α-D-fucopyranoside)-2-hydroxypropane;
39. 1-(C-β-L-fucopyranoside)-2-hydroxypropane;
40. 1-(C-α-L-fucopyranoside)-2-hydroxypropane;
41. 1-(C-β-D-fucopyranoside)-2-aminopropane;
42. 1-(C-α-D-fucopyranoside)-2-aminopropane;
43. 1-(C-β-L-fucopyranoside)-2-aminopropane;
44. 1-(C-α-L-fucopyranoside)-2-aminopropane;
45. 1-(C-β-D-fucopyranoside)-2-phenylaminopropane;
46. 1-(C-α-D-fucopyranoside)-2-phenylaminopropane;
47. 1-(C-β-L-fucopyranoside)-2-phenylaminopropane;
48. 1-(C-α-L-fucopyranoside)-2-phenylaminopropane;
49. ethyl 3-methyl-4-(C-β-D-fucopyranoside)butyrate;
50. ethyl 3-methyl-4-(C-α-D-fucopyranoside)butyrate;
51. ethyl 3-methyl-4-(C-β-L-fucopyranoside)butyrate;
52. ethyl 3-methyl-4-(C-α-L-fucopyranoside)butyrate;
53. 6-(C-β-D-fucopyranoside)-5-ketohexanoic acid;
54. 6-(C-α-D-fucopyranoside)-5-ketohexanoic acid;
55. 6-(C-β-L-fucopyranoside)-5-ketohexanoic acid;
56. 6-(C-α-L-fucopyranoside)-5-ketohexanoic acid;
57. 6-(C-β-D-fucopyranoside)-5-hydroxyhexanoic acid;
58. 6-(C-α-D-fucopyranoside)-5-hydroxyhexanoic acid;
59. 6-(C-β-L-fucopyranoside)-5-hydroxyhexanoic acid;
60. 6-(C-α-L-fucopyranoside)-5-hydroxyhexanoic acid;
61. 6-(C-β-D-fucopyranoside)-5-aminohexanoic acid;
62. 6-(C-α-D-fucopyranoside)-5-aminohexanoic acid;
63. 6-(C-β-L-fucopyranoside)-5-aminohexanoic acid;
64. 6-(C-α-L-fucopyranoside)-5-aminohexanoic acid;
65. 1-(C-β-D-fucopyranoside)hexane-2,6-diol;
66. 1-(C-α-D-fucopyranoside)hexane-2,6-diol;
67. 1-(C-β-L-fucopyranoside)hexane-2,6-diol;
68. 1-(C-α-L-fucopyranoside)hexane-2,6-diol;
69. 5-(C-β-D-fucopyranoside)-4-ketopentanoic acid;
70. 5-(C-α-D-fucopyranoside)-4-ketopentanoic acid;
71. 5-(C-β-L-fucopyranoside)-4-ketopentanoic acid;
72. 5-(C-α-L-fucopyranoside)-4-ketopentanoic acid;
73. 5-(C-β-D-fucopyranoside)-4-hydroxypentanoic acid;
74. 5-(C-α-D-fucopyranoside)-4-hydroxypentanoic acid;
75. 5-(C-β-L-fucopyranoside)-4-hydroxypentanoic acid;
76. 5-(C-α-L-fucopyranoside)-4-hydroxypentanoic acid;
77. 5-(C-β-D-fucopyranoside)-4-aminopentanoic acid;
78. 5-(C-α-D-fucopyranoside)-4-aminopentanoic acid
79. 5-(C-β-L-fucopyranoside)-4-aminopentanoic acid;
80. 5-(C-α-L-fucopyranoside)-4-aminopentanoic acid;
81. 1-(C-β-D-fucopyranoside)pentane-2,5-diol;
82. 1-(C-α-D-fucopyranoside)pentane-2,5-diol;
83. 1-(C-β-L-fucopyranoside)pentane-2,5-diol;
84. 1-(C-α-L-fucopyranoside)pentane-2,5-diol;
85. 1-(C-β-D-glucopyranosyl)-2-hydroxypropane;
86. 1-(C-α-D-glucopyranosyl)-2-hydroxypropane;
87. 1-(C-β-D-glucopyranosyl)-2-aminopropane;
88. 1-(C-α-D-glucopyranosyl)-2-aminopropane;
89. 1-(C-β-D-glucopyranosyl)-2-phenylaminopropane;
90. 1-(C-α-D-glucopyranosyl)-2-phenylaminopropane;
91. ethyl 3-methyl-4-(C-β-D-glucopyranosyl)butyrate;
92. ethyl 3-methyl-4-(C-α-D-glucopyranosyl)butyrate;
93. 6-(C-β-D-glucopyranosyl)-5-ketohexanoic acid;
94. 6-(C-α-D-glucopyranosyl)-5-ketohexanoic acid;
95. 6-(C-β-D-glucopyranosyl)-5-hydroxyhexanoic acid;
96. 6-(C-α-D-glucopyranosyl)-5-hydroxyhexanoic acid;
97. 6-(C-β-D-glucopyranosyl)-5-aminohexanoic acid;
98. 6-(C-α-D-glucopyranosyl)-5-aminohexanoic acid;
99. 6-(C-β-D-glucopyranosyl)-5-phenylaminohexanoic acid;
100. 6-(C-α-D-glucopyranosyl)-5-phenylaminohexanoic acid;
101. 1-(C-β-D-glucopyranosyl)hexane-2,6-diol;
102. 1-(C-α-D-glucopyranosyl)hexane-2,6-diol;
103. 6-(C-β-D-glucopyranosyl)-5-ketopentanoic acid;
104. 6-(C-α-D-glucopyranosyl)-5-ketopentanoic acid;
105. 6-(C-β-D-glucopyranosyl)-5-hydroxypentanoic acid;
106. 6-(C-α-D-glucopyranosyl)-5-hydroxypentanoic acid;
107. 6-(C-β-D-glucopyranosyl)-5-aminopentanoic acid;
108. 6-(C-α-D-glucopyranosyl)-5-hydroxypentanoic acid;
109. 6-(C-β-D-glucopyranosyl)-5-phenylaminopentanoic acid;
110. 6-(C-α-D-glucopyranosyl)-5-phenylaminopentanoic acid;
111. 1-(C-β-D-glucopyranosyl)pentane-2,5-diol;
112. 1-(C-α-D-glucopyranosyl)pentane-2,5-diol;
113. 1-(C-β-D-galactopyranosyl)-2-hydroxypropane;
114. 1-(C-α-D-galactopyranosyl)-2-hydroxypropane;
115. 1-(C-β-D-galactopyranosyl)-2-aminopropane;
116. 1-(C-α-D-galactopyranosyl)-2-aminopropane;
117. 1-(C-β-D-galactopyranosyl)-2-phenylaminopropane;
118. 1-(C-α-D-galactopyranosyl)-2-phenylaminopropane;
119. ethyl 3-methyl-4-(β-D-galactopyranosyl)butyrate;
120. ethyl 3-methyl-4-(α-D-galactopyranosyl)butyrate;
121. 6-(C-β-D-galactopyranosyl)-5-ketohexanoic acid;
122. 6-(C-α-D-galactopyranosyl)-5-ketohexanoic acid;
123. 6-(C-β-D-galactopyranosyl)-5-hydroxyhexanoic acid;
124. 6-(C-α-D-galactopyranosyl)-5-hydroxyhexanoic acid;
125. 6-(C-β-D-galactopyranosyl)-5-aminohexanoic acid;
126. 6-(C-α-D-galactopyranosyl)-5-aminohexanoic acid;
127. 6-(C-β-D-galactopyranosyl)-5-phenylaminohexanoic acid;
128. 6-(C-α-D-galactopyranosyl)-5-phenylaminohexanoic acid;
129. 1-(C-β-D-galactopyranosyl)hexane-2,6-diol;
130. 1-(C-α-D-galactopyranosyl)hexane-2,6-diol;
131. 6-(C-β-D-galactopyranosyl)-5-ketopentanoic acid;
132. 6-(C-β-D-galactopyranosyl)-5-ketopentanoic acid;
133. 6-(C-β-D-galactopyranosyl)-5-hydroxypentanoic acid;
134. 6-(C-α-D-galactopyranosyl)-5-hydroxypentanoic acid;
135. 6-(C-β-D-galactopyranosyl)-5-aminopentanoic acid;
136. 6-(C-α-D-galactopyranosyl)-5-aminopentanoic acid;
137. 6-(C-β-D-galactopyranosyl)-5-phenylaminopentanoic acid;
138. 6-(C-α-D-galactopyranosyl)-5-phenylaminopentanoic acid;
139. 1-(C-β-D-galactopyranosyl)pentane-2,6-diol;
140. 1-(C-α-D-galactopyranosyl)pentane-2,6-diol;
141. 1-(C-β-D-fucofuranosyl)propan-2-one;
142. 1-(C-α-D-fucofuranosyl)propan-2-one;
143. 1-(C-β-L-fucofuranosyl)propan-2-one;
144. 1-(C-α-L-fucofuranosyl)propan-2-one;
145. 3′-(acetamido-C-β-D-glucopyranosyl)propane-2′-one;
146. 3′-(acetamido-C-α-D-glucopyranosyl)propane-2′-one;
147. 1-(acetamido-C-β-D-glucopyranosyl)-2-hydroxylpropane;
148. 1-(acetamido-C-β-D-glucopyranosyl)-2-aminopropane;
149. 1-(acetamido-C-β-D-glucopyranosyl)-2-phenylaminopropane;
150. 1-(acetamido-C-α-D-glucopyranosyl)-2-phenylaminopropane;
151. ethyl 3-methyl-4-(acetamido-C-β-D-glucopyranosyl)butyrate;
152. ethyl 3-methyl-4-(acetamido-C-α-D-glucopyranosyl)butyrate;
153. 6-(acetamido-C-β-D-glucopyranosyl)-5-ketohexanoic acid;
154. 6-(acetamido-C-α-D-glucopyranosyl)-5-ketohexanoic acid;
155. 6-(acetamido-C-β-D-glucopyranosyl)-5-hydroxyhexanoic acid;
156. 6-(acetamido-C-α-D-glucopyranosyl)-5-hydroxyhexanoic acid;
157. 6-(acetamido-C-β-D-glucopyranosyl)-5-aminohexanoic acid;
158. 6-(acetamido-C-α-D-glucopyranosyl)-5-aminohexanoic acid;
159. 6-(acetamido-C-β-D-glucopyranosyl)-5-phenylaminohexanoic acid;
160. 6-(acetamido-C-α-D-glucopyranosyl)-5-phenylaminohexanoic acid;
161. 1-(acetamido-C-β-D-glucopyranosyl)hexane-2,6-diol;
162. 1-(acetamido-C-α-D-glucopyranosyl)hexane-2,6-diol;
163. 6-(acetamido-C-β-D-glucopyranosyl)-5-ketopentanoic acid;
164. 6-(acetamido-C-α-D-glucopyranosyl)-5-ketopentanoic acid;
165. 6-(acetamido-C-β-D-glucopyranosyl)-5-hydroxypentanoic acid;
166. 6-(acetamido-C-α-D-glucopyranosyl)-5-hydroxypentanoic acid;
167. 6-(acetamido-C-β-D-glucopyranosyl)-5-aminopentanoic acid;
168. 6-(acetamido-C-α-D-glucopyranosyl)-5-aminopentanoic acid;
169. 6-(acetamido-C-β-D-glucopyranosyl)-5-phenylaminopentanoic acid;
170. 6-(acetamido-C-α-D-glucopyranosyl)-5-phenylaminopentanoic acid;
171. 1-(acetamido-C-β-D-glucopyranosyl)pentane-2,5-diol;
172. 1-(acetamido-C-α-D-glucopyranosyl)pentane-2,5-diol.

As non-limiting illustrations of C-glycoside derivatives that are more particularly suitable for use in the invention, mention may be made especially of the following derivatives:
C-β-D-xylopyranoside-n-propan-2-one,
C-α-D-xylopyranoside-n-propan-2-one,
C-β-D-xylopyranoside-2-hydroxypropane,
C-α-D-xylopyranoside-2-hydroxypropane,
1-(C-β-D-fucopyranoside)propan-2-one,
1-(C-α-D-fucopyranoside)propan-2-one,
1-(C-β-L-fucopyranoside)propan-2-one,
1-(C-α-L-fucopyranoside)propan-2-one,
1-(C-β-D-fucopyranoside)-2-hydroxypropane,
1-(C-α-D-fucopyranoside)-2-hydroxypropane,
1-(C-β-L-fucopyranoside)-2-hydroxypropane,
1-(C-α-L-fucopyranoside)-2-hydroxypropane,
1-(C-β-D-glucopyranosyl)-2-hydroxylpropane,
1-(C-α-D-glucopyranosyl)-2-hydroxylpropane,
1-(C-β-D-galactopyranosyl)-2-hydroxylpropane,
1-(C-α-D-galactopyranosyl)-2-hydroxylpropane
1-(C-β-D-fucofuranosyl)propan-2-one,
1-(C-α-D-fucofuranosyl)propan-2-one
1-(C-β-L-fucofuranosyl)propan-2-one,
1-(C-α-L-fucofuranosyl)propan-2-one,
C-β-D-maltopyranoside-n-propan-2-one,
C-α-D-maltopyranoside-n-propan-2-one
C-β-D-maltopyranoside-2-hydroxypropane,
C-α-D-maltopyranoside-2-hydroxypropane, isomers thereof and mixtures thereof.

According to one embodiment, C-β-D-xylopyranoside-2-hydroxypropane or C-α-D-xylopyranoside-2-hydroxypropane, and better still C-β-D-xylopyranoside-2-hydroxypropane, may advantageously be used for the preparation of a composition according to the invention.

According to one particular embodiment, the C-glycoside derivative may be C-β-D-xylopyranoside-2-hydroxypropane in the form of a solution containing 30% by weight of active material in a water/propylene glycol mixture (60%/40% by weight), such as the product manufactured by Chimex under the trade name Mexoryl SBB®.

Needless to say, according to the invention, a C-glycoside derivative corresponding to formula (I) may be used alone or as a mixture with other C-glycoside derivatives and in all proportions.

A C-glycoside derivative that is suitable for use in the invention may especially be obtained via the synthetic method described in document WO 02/051 828.

The amount of C-glycoside derivative to be used in a composition according to the invention depends on the desired cosmetic or therapeutic effect, and may thus vary within a wide range.

A person skilled in the art can readily determine the appropriate amounts, on the basis of his general knowledge.

A composition according to the invention may comprise a C-glycoside derivative in a proportion of about from 0.0001% to about 25% by weight of active material relative to the total weight of the composition, in particular from about 0.001% to about 10% by weight of active material and more particularly from about 0.05% to about 5% by weight of C-glycoside derivative active material relative to the total weight of the composition.

The C-glycoside derivative is preferably used according to the invention for the preparation of a composition formulated for topical administration, i.e. containing a cosmetically or dermatologically acceptable medium, i.e. a medium that is compatible with the skin, the nails, mucous membranes, fabrics, the scalp and/or the hair.

It is also clear that the effective amount of active agent corresponds to the amount necessary to obtain the desired result, and that the formulation of the compositions according to the invention depends on the use for which these compositions are intended.

In particular, two major categories of compositions according to the invention may be distinguished, as a function of the conditions under which they will be applied to the skin.

The first category corresponds to cosmetic compositions, i.e. compositions intended to be applied to healthy skin in order to improve the aesthetics and especially the comfort. Healthy skin is defined by the absence of pathologies such as infections, inflammation, erythema, or injuries such as burns or cuts. However, in this definition, healthy skin does not mean skin in perfect condition.

In particular, healthy skin may show signs of dryness that may be of exogenous origin (the skin becomes dry, for example, during exposure to dry and very cold air) or of endogenous physiological origin (for example at the moment of the menopause-related drop in hormone levels).

In one particular embodiment of the invention, a C-glycoside derivative is used for the preparation of a composition formulated for cosmetic use, for example on fragile skin (for example baby skin), depleted skin (for example the skin of a very elderly person, or the skin of a person for whom the composition of the hydro-lipid film of the skin is modified), or sensitive skin. For this use, the C-glycoside derivative may be formulated in a composition as described above.

The cosmetic composition is thus formulated to be applied to the skin and comprises at least one C-glycoside derivative, the said composition making it possible to prevent, resorb and/or make disappear the said signs of cutaneous discomfort associated with deficiency of the barrier function.

According to the invention, the C-glycoside derivative may also be used for the preparation of a therapeutic composition for improving the condition of damaged skin, such as atopic skin.

The damaged skin under consideration may be of any origin, for example infectious, allergic, nervous or traumatic. A therapeutic composition may be applied directly to the damaged area or to its vicinity, or alternatively, in the case of pathologies showing crisis periods and rest periods, at a site that has been damaged, as a preventive measure, to prolong a state of remission between two crises.

Thus, the invention also relates to the use of at least one C-glycoside derivative for the preparation of a composition formulated for therapeutic use, for example to damaged skin such as atopic skin. It more particularly relates to a dermatological method for improving and/or reinforcing the barrier function of damaged skin, such as atopic skin comprising at least a step of applying to the skin a composition comprising at least one C-glycoside derivative.

The composition will then be formulated with a pharmaceutically acceptable vehicle.

According to one preferred embodiment of the invention, the composition has a pH close to that of the skin, of between 4 and 7.

When it is applied topically, the composition comprising at least one C-glycoside derivative may be applied to the face, the neck, the scalp, mucous membranes and the nails or any other area of bodily skin, including the hands and feet.

The compositions according to the invention are preferably formulated in a form suitable for topical administration.

They comprise a physiologically acceptable medium.

The term “physiologically acceptable medium” is intended to denote a medium that is compatible with human keratin materials and/or fibres, for instance, in a non-limiting manner, the skin, mucous membranes, the nails, the scalp and/or the hair.

This physiologically acceptable medium comprises water, optionally mixed with one or more organic solvents such as C₁-C₈ alcohols, especially ethanol, isopropanol, tert-butanol, n-butanol, polyols, for instance glycerol, propylene glycol or butylene glycol, and polyol ethers.

They are especially in the form of aqueous, aqueous-alcoholic or oily solutions, dispersions of the lotion or serum type, anhydrous or oily gels, emulsions of liquid or semi-liquid consistency of the milk type, obtained by dispersing a fatty phase in an aqueous phase (O/W) or conversely (W/O), suspensions or emulsions of soft, semi-solid or solid consistency of the cream or gel type, microemulsions, or alternatively microcapsules, microparticles or vesicular dispersions of ionic and/or nonionic type. These compositions are prepared according to the usual methods.

The amounts of the various constituents of the compositions used according to the invention are those conventionally used in the fields under consideration.

These compositions especially constitute protective, treatment or care creams for the face, for the hands or for the body, protective or care body milks, lotions, gels or mousses for caring for the skin and mucous membranes or for cleansing the skin, masks or patches.

The compositions may also consist of solid preparations constituting cleansing soaps or bars.

The compositions used according to the invention may also contain one or more adjuvants that are common in cosmetics and dermatology, such as oils, hydrophilic or lipophilic gelling agents, hydrophilic or lipophilic active agents, chelating agents, preserving agents, antioxidants, solvents, fragrances, fillers and dyestuffs.

The amounts of these various adjuvants are those conventionally used in the fields under consideration, for example from 0.01% to 20% of the total weight of the composition.

Needless to say, person skilled in the art will take care to select this or these optional additives and/or the amounts thereof such that the advantageous properties intrinsically associated with the composition in accordance with the invention are not, or are not substantially, adversely affected by the envisaged addition(s).

As oils that may be used in the invention, mention may be made of mineral oils (liquid petroleum jelly), plant oils (shea butter oil or sweet almond oil), animal oils, synthetic oils, silicone oils (cyclomethicone) and fluoro oils (perfluoropolyethers). Fatty alcohols, fatty acids (stearic acid) and waxes (paraffin wax, carnauba wax or beeswax) may also be used as fatty substance.

As emulsifiers that may be used in the invention, mention may be made of Polysorbate 60 and sorbitan stearate sold, respectively, under the trade names Tween 60 and Span 60 by the company ICI. Co-emulsifiers may be added thereto, such as PPG-3 myristyl ether sold under the trade name Emcol 249-3K by the company Witco.

As solvents that may be used in the invention, mention may be made of water, lower alcohols, especially ethanol and isopropanol, and propylene glycol.

Hydrophilic gelling agents that may be mentioned include carboxyvinyl polymers (carbomer), acrylic copolymers such as acrylate/alkylacrylate copolymers, polyacrylamides, polysaccharides such as hydroxypropylcellulose, natural gums (xanthan gum) and clays, and lipophilic gelling agents that may be mentioned include modified clays, for instance bentones, metal salts of fatty acids, for instance aluminium stearates, hydrophobic silica, polyethylenes and ethylcellulose.

Hydrophilic active agents that may be used include proteins or protein hydrolysates, amino acids, polyols, urea, allantoin, sugars and sugar derivatives, water-soluble vitamins, starch, and bacterial or plant extracts, especially extracts of Aloe vera.

Lipophilic active agents that may be used include tocopherol (vitamin E) and derivatives thereof, essential fatty acids, ceramides and essential oils.

It is also possible to add to the composition according to the invention one or more additional hydrophilic or lipophilic UV-A-active and/or UV-B-active sunscreens, optionally comprising a sulfonic function. The sunscreen is preferably chosen from organic screening agents and/or mineral screening agents.

Organic screening agents that may especially be mentioned include cinnamic derivatives, salicylic derivatives, camphor derivatives, triazine derivatives, benzophenone derivatives, dibenzoylmethane derivatives, β,β-diphenylacrylate derivatives, p-amino-benzoic acid derivatives, the screening polymers and screening silicones described in patent application WO 93/04665, or the organic screening agents described in patent application EP-A-0 487 404.

Mineral screening agents that may especially be mentioned include pigments or alternatively nanopigments with a mean primary particle size generally of between 5 nm and 100 nm and preferably between 10 and 50 nm, of coated or uncoated metal oxides, for instance pigments of titanium oxide (amorphous or crystallized in rutile and/or anatase form), of iron oxide, of zinc oxide, of zirconium oxide or of cerium oxide, which are all photoprotective agents that are well known per se, which act by physical blocking (reflection and/or scattering) of UV radiation. Standard coating agents are, moreover, alumina and/or aluminium stearate. Such coated or uncoated metal oxide pigments are described in particular in patent applications EP-A-0 518 772 and EP-A-0 518 773.

Examples of additional UV-A-active and/or UV-B-active sunscreens that may be mentioned include:
p-aminobenzoic acid,
oxyethylenated (25 mol) p-aminobenzoate,
2-ethylhexyl p-dimethylaminobenzoate,
N-oxypropylenated ethyl p-aminobenzoate,
glyceryl p-aminobenzoate,
homomethyl salicylate,
2-ethylhexyl salicylate,
triethanolamine salicylate,
4-isopropylbenzyl salicylate,
4-tert-butyl-4′-methoxydibenzoylmethane (Parsol 1789 from Givaudan-Roure),
2-ethylhexyl p-methoxycinnamate (Parsol MCX from Givaudan-Roure),
4-isopropyldibenzoylmethane (Eusolex 8020 from Merck),
menthyl anthranilate,
2-ethylhexyl 2-cyano-3,3′-diphenylacrylate (Uvinul N539 from BASF),
ethyl 2-cyano-3,3′-diphenylacrylate,
2-phenylbenzimidazole-5-sulfonic acid and salts thereof,
3-(4′-trimethylammonium)benzylidenebornan-2-one methyl sulfate,
2-hydroxy-4-methoxybenzophenone (Uvinul MS 40 from BASF),
2-hydroxy-4-methoxybenzophenone 5-sulfonate (Uvinul MS 40 from BASF),
2,4-dihydroxybenzophenone (Uvinul 400 from BASF),
2,2′,4,4′-tetrahydroxybenzophenone (Uvinul D 50 from BASF),
2,2′-dihydroxy-4,4′-dimethoxybenzophenone (Helisorb II from Norquay),
2-hydroxy-4-n-octoxybenzophenone,
2-hydroxy-4-methoxy-4′-methylbenzophenone,
α-(2-oxoborn-3-ylidene)tolyl-4-sulfonic acid and salts thereof,
3-(4′-sulfo)benzylidenebornan-2-one and salts thereof,
3-(4′-methylbenzylidene)-d,l-camphor,
3-benzylidene-d,l-camphor,
benzene-1,4-bis(3-methylidene-10-camphorsulfonic acid) and salts thereof (Mexoryl SX from Chimex),
urocanic acid,
2,4,6-tris[p-(2′-ethylhexyl-1′-oxycarbonyl)anilino]-1,3,5-triazine,
2-[p-(tert-butylamido)anilino]-4,6-bis[p-(2′-ethylhexyl-1′-oxycarbonyl)anilino]-1,3,5-triazine,
2,4-bis{4-[2-ethylhexyloxy]-2-hydroxyphenyl}-6-(4-methoxyphenyl)-1,3,5-triazine,
the polymer of N-(2 and 4)[(2-oxoborn-3-ylidene)methyl)benzyl]acrylamide,
4,4-bis(benzimidazolyl)phenylene-3,3′,5,5′-tetrasulfonic acid and salts thereof,
2,2′-methylenebis[6-(2H-benzotriazol-2-yl)-4-(1,1,3,3-tetramethylbutyl)phenol],
polyorganosiloxanes containing a malonate function.

The invention also relates to a cosmetic skin treatment process for improving and/or reinforcing the skin's barrier function, comprising at least one step that consists in applying to the skin at least one composition containing at least one C-glycoside derivative, especially as defined above.

According to a first mode, the process according to the invention comprises at least one step that consists in applying to the skin of people with fragile or delicate skin at least one composition comprising at least one C-glycoside derivative, especially as defined above.

According to another embodiment, it comprises at least one step that consists in applying to the skin of people with depleted skin, in particular the skin of people at least 60 years old, or even at least 75 years old, at least one composition comprising at least one C-glycoside derivative, especially as defined above.

According to another embodiment, it comprises at least one step that consists in applying to the skin of people with aggravated skin or an area of aggravated skin, in particular shaved facial or bodily skin, at least one composition comprising at least one C-glycoside derivative, especially as defined above.

Another mode also consists in applying to the skin of people with sensitive skin at least one composition comprising at least one C-glycoside derivative, especially as defined above.

The examples featured hereinbelow present the results of in vitro studies relating to the effect of a C-glycoside derivative on the barrier function of a reconstructed human epidermis, and also non-limiting examples of compositions according to the invention that are useful for improving and/or reinforcing the barrier function.

### EXAMPLES

The C-glycoside derivative used is C—β-D-xylopyranoside-2-hydroxypropane, sold under the name Mexoryl® from Chimex. It is in the form of a solution containing 30% by weight of active material in a 60/40 water/1,2-propanediol mixture.

### Example 1

### Study of the Penetration of Caffeine on a Reconstructed Human Skin

The efficacy of the barrier function of a skin may be evaluated by measuring the passage through it of a labelled molecule, for instance radiolabelled caffeine.

Skin with an improved barrier function is thus characterized in that it slows down, or even reduces, the passage of the said molecule through its epidermis.

Two batches of reconstructed epidermis (Episkin J13) (batches 1 and 2) are treated for 5 days with the product C-β-D-xylopyranoside-n-propan-2-one, which is applied in the culture medium at a final concentration of 0.2% and 0.04% by weight/volume.

Vitamin C is used as reference molecule at a concentration of 200 μg/ml, in systemic treatment.

After 5 days of culturing, 100 μl and 2 UCi/ml, i.e. 0.04 mM of [¹⁴C]-caffeine (radiolabelled caffeine) and 0.35 mM of cold caffeine (non-radiolabelled caffeine) are applied to the surface, and kinetics of uptake into the medium are determined at eight different times.

The results are expressed as a percentage of passage relative to an untreated control.

| | | Batch 1 of reconstructed | | | Batch 2 of reconstructed | | |
|---|---|---|---|---|---|---|---|
| | | epidermis | | | epidermis | | |
| | | | C-β-D- | | | C-β-D- | |
| | | | xylopyranoside- | | | xylopyranoside- | |
| | | | n-propan-2-one | | | n-propan-2-one | |
| Sample taken | | Vitamin C | 0.2% | 0.04% | Vitamin C | 0.2% | 0.04% |
| at time T = | Control | 200 μg/ml | AM | AM | 200 μg/ml | AM | AM |
| 1 h | 100 | — | | | 117 | 162 | 143 |
| 1 h 30 | 100 | 81 | 66 | 52 | — | — | — |
| 2 h | 100 | — | — | — | 77 * | 61 * | 65 * |
| 3 h | 100 | 95 | 94 | 66 | 84 | 82 | 92 |
| 4 h | 100 | 81 | 59 * | 64 * | 86 * | 67 * | 78 * |
| 5 h | 100 | 80 * | 63 * | 73 * | 71 * | 75 * | 84 |
| 6 h | 100 | 86 | 63 * | 70 * | 94 | 75 * | 80 * |
| 7 h | 100 | 75 * | 66 * | 69 * | 95 | 84 * | 91 |
| 8 h | 100 | — | — | — | 96 | 80 * | 88 * |
| 9 h | 100 | 85 | 79 * | 77 * | 85 | 88 | 93 |
| 10 h | 100 | — | — | — | 95 | 80 * | 85 * |
| 24 h | 100 | 85 | 70 * | 75 * | 99 | 95 | 99 |

*: statistically significant values relative to the control
AM: active material

Under these conditions, the product C-β-D-xylopyranoside-n-propan-2-one slows down the passage of the radiolabelled caffeine through the reconstructed epidermides, this reflecting an improvement in the barrier function of the reconstructed epidermides.

This slowing down is greater with the product C-β-D-xylopyranoside-n-propan-2-one than with vitamin C, used as reference molecule in this test.

The results were validated on two different batches of reconstructed epidermis.

### Example 2

### Night Cream

| | % by |
|---|---|
| Name | weight |
| Glyceryl stearate SE | 3.00 |
| Myristyl alcohol | 0.10 |
| Stearyl alcohol | 0.10 |
| Sodium hydroxide | 0.05 |
| Myristyl myristate | 2.00 |
| Cetyl alcohol | 3.80 |
| Cyclopentasiloxane | 5.00 |
| Myristic acid | 0.036 |
| Palmitic acid | 0.528 |
| Stearic acid | 0.636 |
| Hydrogenated polyisobutene | 8.50 |
| Candelilla wax | 0.90 |
| Biotin | 0.04 |
| Petroleum jelly | 4.00 |
| PEG-40 stearate (Myrj 52 P) | 2.00 |
| Moisturizers | qs |
| C-β-D-Xylopyranoside-2-hydroxypropane at 30% by | 3% (AM) |
| weight of active material in a 60/40 water/1,2- | |
| propanediol mixture | |
| Conservateurs | qs |
| Eau | qs 100 |

AM: active material

### Example 4

### Serum

| | % by |
|---|---|
| Name | weight |
| Butylene glycol | 5.00 |
| Xanthan gum | 0.25 |
| Stearyl alcohol | 1.30 |
| Laureth-7 | 0.07 |
| C-β-D-Xylopyranoside-2-hydroxypropane at 30% by | 1.80% (AM) |
| weight of active material in a 60/40 water/1,2- | |
| propanediol mixture | |
| Cyclohexasiloxane | 10.00 |
| Glycerol | 5.00 |
| PEG-20 methyl glucose sesquistearate | 2.00 |
| Ceteareth-20 | 0.70 |
| C13-14 Isoparaffin | 0.21 |
| Prunus armeniaca (apricot) kernel oil | 6.00 |
| Methyl glucose sesquistearate | 2.00 |
| Polyacrylamide | 0.40 |
| Shorea robusta seed butter | 2.00 |
| Venuceane from Sederma (Thermus thermophilus ferment) | 2.00 |
| Preserving agents | qs |
| Water | qs 100 |

AM: active material

### Example 5

### Day Cream

| | % by |
|---|---|
| Ingredients | weight |
| Stearic acid | 3.0 |
| Mixture of glyceryl monostearate and polyethylene glycol | 2.5 |
| stearate (100 EO) | |
| Polyethylene glycol stearate (20 EO) | 1.0 |
| Cyclohexadimethylsiloxane | 10 |
| Plant oils | 7 |
| Synthetic oils | 6 |
| Fillers | 3 |
| Preserving agents | qs |
| Oxyethylenated (16 EO) dimethylsiloxane containing methoxy | 1.0 |
| end groups | |
| Silicone gum | 0.2 |
| Acrylic copolymer as an inverse emulsion (Simulgel 600 from | 1.7 |
| SEPPIC) | |
| Stearyl alcohol | 1.0 |
| C-β-D-Xylopyranoside-2-hydroxypropane at 30% by weight of | 10** |
| active material in a 60/40 water/1,2-propanediol mixture | |
| Water | qs 100 |

**expressed as a weight percentage of commercial solution

These compositions are prepared in a manner that is conventional for a person skilled in the art.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A cosmetic method for improving and/or reinforcing the skin's barrier function comprising at least a step of applying to the skin a composition comprising at least one C-glycoside derivative.

2. A cosmetic method for improving and/or reinforcing the protection of the skin against external attack comprising at least a step of applying to the skin a composition comprising at least one C-glycoside derivative.

3. The cosmetic method according to claim 1, wherein the said C-glycoside derivative and/or the said composition is intended for preventing and/or reducing the cutaneous discomfort of a skin type.

4. The cosmetic method according to claim 2, wherein the said C-glycoside derivative and/or the said composition is intended for preventing and/or reducing the cutaneous discomfort of a skin type.

5. The method according to claim 3, wherein said cutaneous discomfort is induced by exogenous stress of chemical, environmental or mechanical origin and/or endogenous stress.

6. The method according to claim 4, wherein said cutaneous discomfort is induced by exogenous stress of chemical, environmental or mechanical origin and/or endogenous stress.

7. The method according to claim 3, wherein the cutaneous discomfort is characterized by tautness, stinging, sensations of heating and/or itching.

8. The method according to claim 4, wherein the cutaneous discomfort is characterized by tautness, stinging, sensations of heating and/or itching.

9. The method according to claim 1, wherein the said C-glycoside derivative and/or the said composition is intended for improving and/or reinforcing the barrier function of a skin type chosen from fragile skin, depleted skin, aggravated skin and/or sensitive skin.

10. A dermatological method for improving and/or reinforcing the barrier function of damaged skin comprising at least a step of applying to the skin a composition comprising at least one C-glycoside derivative.

11. The method according to claim 10, wherein said damaged skin is an atopic skin.

12. The method according to claim 10, intended for prolonging the remission phases between acute crises of dermatological complaints.

13. The method according to claim 1, wherein the C-glycoside derivative corresponds to the general formula (I) below: 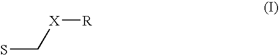
in which:
R represents:
a saturated C₁-C₂₀ or unsaturated C₂-C₂₀ linear alkyl radical, or a saturated or unsaturated, branched or cyclic C₃-C₂₀ alkyl radical;
a saturated C₁-C₂₀ or unsaturated C₂-C₂₀ or saturated or unsaturated, branched or cyclic C₃-C₂₀ linear hydrofluoroalkyl or perfluoroalkyl radical;
the hydrocarbon-based chain constituting the said radicals possibly being, where appropriate, interrupted with 1, 2, 3 or more heteroatoms chosen from:
an oxygen,
a sulfur,
a nitrogen, and
a silicon,
and possibly being optionally substituted with at least one radical chosen from:
—OR₄,
—SR₄,
—NR₄R₅,
—COOR₄,
—CONHR₄,
—CN,
a halogen atom,
a C₁-C₆ hydrofluoroalkyl or perfluoroalkyl radical, and/or
a C₃-C₈ cycloalkyl radical,
with R₄ and R₅ possibly representing, independently of each other, a hydrogen atom or a saturated C₁-C₃₀ or unsaturated C₂-C₃₀, or a saturated or unsaturated, branched or cyclic C₃-C₃₀ linear alkyl, perfluoroalkyl or hydrofluoroalkyl radical; or a C₆-C₁₀ aryl radical,
X represents a radical chosen from the groups: 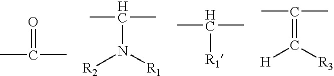
with R₁, R₂ and R₃ representing, independently of each other, a hydrogen atom or a radical R, with R as defined above, and R′₁ represents a hydrogen atom, an —OH group or a radical R as defined above, R₁ possibly also denoting a C₆-C₁₀ aryl radical;
S represents a monosaccharide or a polysaccharide comprising up to 20 sugar units, in pyranose and/or furanose form and of L and/or D series, the said mono- or polysaccharide possibly being substituted with a mandatorily free hydroxyl group, and optionally one or more optionally protected amine function(s), and
the bond S—CH₂—X represents a bond of C-anomeric nature, which may be a or and also the cosmetically acceptable salts thereof, the solvates thereof such as hydrates, and the isomers thereof.

14. The method according to claim 13, in which S represents a monosaccharide chosen from D-glucose, D-xylose, L-fucose, D-galactose and D-maltose.

15. The method according to claim 14, in which X represents a group chosen from —CO—, —CH(OH)— and —CH(NH₂)— group.

16. The method according to claim 15, in which R denotes a linear C₁-C₄, optionally substituted with —OH, —COOH or —COOR″₂, R″₂ being a saturated C₁-C₄ alkyl radical.

17. The method according to claim 1, in which the C-glycoside derivative is chosen from:
C-β-D-xylopyranoside-n-propan-2-one,
C-α-D-xylopyranoside-n-propan-2-one,
C-β-D-xylopyranoside-2-hydroxypropane,
C-α-D-xylopyranoside-2-hydroxypropane,
1-(C-β-D-fucopyranoside)propan-2-one,
1-(C-α-D-fucopyranoside)propan-2-one,
1-(C-β-L-fucopyranoside)propan-2-one,
1-(C-α-L-fucopyranoside)propan-2-one,
1-(C-β-D-fucopyranoside)-2-hydroxypropane,
1-(C-α-D-fucopyranoside)-2-hydroxypropane,
1-(C-β-L-fucopyranoside)-2-hydroxypropane,
1-(C-α-L-fucopyranoside)-2-hydroxypropane,
1-(C-β-D-glucopyranosyl)-2-hydroxylpropane,
1-(C-α-D-glucopyranosyl)-2-hydroxylpropane,
1-(C-β-D-galactopyranosyl)-2-hydroxylpropane,
1-(C-α-D-galactopyranosyl)-2-hydroxylpropane
1-(C-β-D-fucofuranosyl)propan-2-one,
1-(C-α-D-fucofuranosyl)propan-2-one
1-(C-β-L-fucofuranosyl)propan-2-one,
1-(C-α-L-fucofuranosyl)propan-2-one,
C-β-D-maltopyranoside-n-propan-2-one,
C-α-D-maltopyranoside-n-propan-2-one
C-β-D-maltopyranoside-2-hydroxypropane,
C-α-D-maltopyranoside-2-hydroxypropane, isomers thereof and mixtures thereof.

18. The method according to claim 1, in which the C-glycoside derivative is chosen from C-β-D-xylopyranoside-2-hydroxypropane and C-α-D-xylopyranoside-2-hydroxypropane.

19. The method according to claim 1, wherein the C-glycoside derivative is present in a proportion of about from 0.0001% to about 25% by weight of C-glycoside derivative active material relative to the total weight of the composition.

20. The method according to claim 1, wherein the composition is formulated for topical administration.

21. Cosmetic skin treatment process for improving and/or reinforcing the skin's barrier function, comprising at least one step that consists in applying to the skin of people with fragile or delicate skin at least one composition comprising at least one C-glycoside derivative.

22. Cosmetic skin treatment process for improving and/or reinforcing the skin's barrier function, comprising at least one step that consists in applying to the skin of people at least 60 years old at least one composition comprising at least one C-glycoside derivative.

23. Cosmetic skin treatment process for improving and/or reinforcing the skin's barrier function, comprising at least one step that consists in applying to the skin of people with shaved facial or bodily skin, at least one composition comprising at least one C-glycoside derivative.

24. Cosmetic skin treatment process for improving and/or reinforcing the skin's barrier function, comprising at least one step that consists in applying to the skin of people with sensitive skin at least one composition comprising at least one C-glycoside derivative.
